(19) United States
(12) Patent Application Publication
(10) Pub. No.: US 2026/0061026 A1
(43) Pub. Date: Mar. 5, 2026

(54) COMBINATION OF INOSITOL PHOSPHORYLCERAMIDE SYNTHETASE INHIBITOR AND AMPHOTERICIN B, AND USE THEREOF

(71) Applicant: INSTITUTE OF MICROBIOLOGY, CHINESE ACADEMY OF SCIENCES, Beijing (CN)

(72) Inventors: Linqi WANG, Beijing (CN); Leixin YE, Beijing (CN); Lei CHEN, Beijing (CN); Xiuyun TIAN, Beijing (CN); Lanyue ZHANG, Beijing (CN)

(21) Appl. No.: 19/273,167

(22) Filed: Jul. 18, 2025

Related U.S. Application Data
(63) Continuation of application No. PCT/CN2024/071853, filed on Jan. 11, 2024.

(30) Foreign Application Priority Data
Jan. 20, 2023 (CN) 202310101287.6

(51) Int. Cl. A61K 38/15; A61K 9/127; A61K 31/513; A61K 31/7048; A61P 31/10

(52) U.S. Cl. CPC A61K 38/15; A61K 9/127; A61K 31/513; A61K 31/7048; A61P 31/10

(57) ABSTRACT

Provided are a combination of an inositol phosphorylceramide synthase inhibitor and amphotericin B and use thereof. The use includes preparing a pharmaceutical composition, which includes an inositol phosphorylceramide synthetase inhibitor and amphotericin B or a salt thereof. Inositol phosphorylceramide is an important and conserved component of fungal plasma membranes. In the pharmaceutical composition according to an embodiment of the present disclosure, due to the presence of the inositol phosphorylceramide synthase inhibitor, the interaction between the inositol phosphorylceramide synthase inhibitor and amphotericin B not only enhances the inhibitory ability of the inositol phosphorylceramide synthase inhibitor on the synthesis of inositol phosphorylceramide in Cryptococcus, but also effectively reduces the dosage of amphotericin B and prolongs the half-life of amphotericin B, facilitating to reduce the adverse reactions of amphotericin B. The pharmaceutical composition exhibits superior fungicidal effect and safety compared to the currently used clinical combination of 5-fluorocytosine and amphotericin B.

### CROSS-REFERENCE TO RELATED APPLICATIONS

The present disclosure is a continuation of International Patent Application No. PCT/CN2024/071853 filed on Jan. 11, 2024, which claims priority to and benefits of patent application No. 202310101287.6, filed with China National Intellectual Property Administration on Jan. 20, 2023, the entire contents of which are incorporated herein by reference.

### FIELD

The present disclosure relates to the field of pharmaceuticals, and more particularly, to combination of an inositol phosphorylceramide synthase inhibitor and amphotericin B and use thereof.

### BACKGROUND

Cryptococcus neoformans is one of the most important human fungal pathogens and serves as a model organism for environmental fungal pathogens. It causes more than 600,000 deaths each year, and even with antifungal treatment, the mortality rate remains above 20%. The current preferred treatment option is a combination of amphotericin B (AmB) approved in the 1950s and 5-fluorocytosine (5-FC) approved in the 1960s. Clinically, despite the administration of effective antifungal agents at effective dosages, invasive fungi still frequently cause recurrent and persistent infections, resulting in chronic illness and consistently high mortality rates.

However, for the treatment of cryptococcal meningitis, amphotericin B remains the only available fungicidal antifungal agent. It can quickly kill Cryptococcus from the central nervous system and reduce the fungal burden in the cerebrospinal fluid, thereby effectively controlling intracranial hypertension of the patients. Therefore, treatment of cryptococcal meningitis still needs to be centered around amphotericin B, and it is crucial to identify compounds that can effectively eliminate drug-resistant fungal strains in the host when used in combination with amphotericin B.

The sphingolipid pathway has been demonstrated to be an important target for the development and application of antifungal agents. However, the sphingolipid components are diverse and complex, and the specific components contributing to drug resistance remain unclear. Inositol phosphorylceramide (IPC) is an essential and conserved component of fungal plasma membranes and also plays an essential role in the pathogenicity of fungal pathogens. AbA inhibits the activity of IPC synthase encoded by the AUR1 gene in yeast, and exhibits certain antifungal activity against yeast-form fungi such as Candida albicans, Saccharomyces cerevisiae, and Cryptococcus neoformans.

### SUMMARY

The present disclosure is based on the inventor's discovery of the following issues and facts.

During the treatment of cryptococcal meningitis, the preferred option for patients in the induction phase involves the administration of low-dose amphotericin B (0.5 to 0.7 mg·kg⁻¹·d⁻¹) in combination with 5-fluorocytosine (100 mg kg⁻¹·d⁻¹). However, amphotericin B is prone to causing adverse reactions in different populations, necessitating close monitoring of hepatotoxicity and nephrotoxicity during treatment, which limits the time and scope of its clinical use. Inositol phosphorylceramide synthetase inhibitors, such as aureobasidin A, exhibit extremely low fungicidal activity against Cryptococcus when used alone. In the present disclosure, the inventors unexpectedly discovered that, the combination of an inositol phosphorylceramide synthetase inhibitor and amphotericin B effectively enhances the fungicidal activity of the inositol phosphorylceramide synthase inhibitor against Cryptococcus, and allows for a significant reduction in the required dose of amphotericin B, a prolonged in vivo retention time and thus an improved therapeutic efficacy and fungicidal activity, as well as reduced adverse effects during treatment. In addition, the combination of inositol phosphorylceramide synthetase inhibitors and amphotericin B exhibit higher pharmacological efficacy and safety compared to the clinically used combination of 5-fluorocytosine and amphotericin B.

In a first aspect of the present disclosure, the present disclosure provides a pharmaceutical composition. According to an embodiment of the present disclosure, the pharmaceutical composition includes: an inositol phosphorylceramide synthetase inhibitor and amphotericin B or a salt thereof. Inositol phosphorylceramide is an important and conserved component of fungal plasma membranes; however, when used alone, inositol phosphorylceramide synthase inhibitors, such as aureobasidin A, exhibit extremely low fungicidal activity against Cryptococcus. In the pharmaceutical composition according to an embodiment of the present disclosure, due to the presence of the inositol phosphorylceramide synthase inhibitor, the interaction between the inositol phosphorylceramide synthase inhibitor and amphotericin B not only enhances the inhibitory ability of the inositol phosphorylceramide synthase inhibitor on the synthesis of inositol phosphorylceramide in Cryptococcus, but also effectively prolongs the pharmacological action of amphotericin B or its salt in the body, leading to increased exposure levels in the body and higher contents of amphotericin B or its salt in brain tissue, thereby reducing the adverse reactions. As a result, both the efficacy and safety of amphotericin B or its salt are significantly improved, thereby effectively killing Cryptococcus and treating or preventing diseases caused by Cryptococcus, such as cryptococcal meningitis. In addition, it has been verified by experiments that the fungicidal effect and safety of the pharmaceutical composition are superior to those of the currently used clinical combination of 5-fluorocytosine and amphotericin B.

According to an embodiment of the present disclosure, the above-mentioned pharmaceutical composition may further include at least one of the following additional technical features.

According to an embodiment of the present disclosure, the inositol phosphorylceramide synthase inhibitor includes a cyclic depsipeptide antibiotic. According to some specific embodiments of the present disclosure, when the inositol phosphorylceramide synthase inhibitor is a cyclic depsipeptide antibiotic, the pharmaceutical composition exhibits enhanced fungicidal activity against Cryptococcus, reduced adverse effects, and improved efficacy and safety compared to the currently used clinical combination of amphotericin B and 5-flucytosine.

According to an embodiment of the present disclosure, the cyclic depsipeptide antibiotic includes at least one of aureobasidin A, enniatin, destruxin, emodepside, or fusafungine.

According to an embodiment of the present disclosure, the cyclic depsipeptide antibiotic includes aureobasidin A. According to some specific embodiments of the present disclosure, when the cyclic depsipeptide antibiotic is aureobasidin A, the pharmaceutical composition exhibits significantly enhanced fungicidal activity against Cryptococcus, further reduced adverse effects, and markedly improved efficacy and safety compared to the currently used clinical combination of amphotericin B and 5-flucytosine.

According to an embodiment of the present disclosure, the salt of amphotericin B includes at least one of: sulfate, phosphate, citrate, tartrate, fumarate, benzoate, adipate, succinate, mesylate, deoxycholate, or maleate of amphotericin B.

According to an embodiment of the present disclosure, a mass ratio of the inositol phosphorylceramide synthase inhibitor to amphotericin B or the salt thereof ranges from 1:3 to 5:1. According to some specific embodiments of the present disclosure, the mass ratio of the inositol phosphorylceramide synthase inhibitor to amphotericin B or the salt thereof has a certain influence on the overall efficacy and safety of the pharmaceutical composition. When the mass ratio of the inositol phosphorylceramide synthase inhibitor to amphotericin B or the salt thereof ranges from 1:3 to 5:1, the pharmaceutical composition exhibits enhanced fungicidal activity against Cryptococcus, reduced adverse effects, and improved efficacy and safety compared to the currently used clinical combination of amphotericin B and 5-flucytosine.

According to an embodiment of the present disclosure, the mass ratio of the inositol phosphorylceramide synthase inhibitor to amphotericin B or the salt thereof ranges from 1:2 to 4:1.

According to an embodiment of the present disclosure, the mass ratio of the inositol phosphorylceramide synthase inhibitor to amphotericin B or the salt thereof is 1:2, 1:1, 2:1, or 4:1. According to some specific embodiments of the present disclosure, the mass ratio of the inositol phosphorylceramide synthase inhibitor to amphotericin B or the salt thereof has a certain influence on the overall efficacy and safety of the pharmaceutical composition. When the mass ratio of the inositol phosphorylceramide synthase inhibitor to amphotericin B or the salt thereof is 1:2, 1:1, 2:1, or 4:1, the pharmaceutical composition exhibits significantly enhanced fungicidal activity against Cryptococcus, further reduced adverse effects, and markedly improved efficacy and safety compared to the currently used clinical combination of amphotericin B and 5-flucytosine.

According to an embodiment of the present disclosure, the pharmaceutical composition further includes a delivery carrier. In some preferred embodiments, the pharmaceutical composition is delivered using a delivery carrier. Preferably, the pharmaceutical composition is delivered to the brain via a brain-targeting ligand.

According to an embodiment of the present disclosure, the delivery carrier includes a liposome. The number of the liposome is one.

According to an embodiment of the present disclosure, the liposome includes a phospholipid.

According to an embodiment of the present disclosure, the phospholipid includes lecithin.

According to an embodiment of the present disclosure, the lecithin includes at least one of soybean lecithin or egg yolk lecithin.

According to an embodiment of the present disclosure, the liposome further includes a brain-targeting ligand.

According to an embodiment of the present disclosure, the brain-targeting ligand includes glutathione. The glutathione is capable of targeting the brain, thereby achieving brain-targeted delivery of the pharmaceutical composition.

According to an embodiment of the present disclosure, a mass ratio of the phospholipid to the brain-targeting ligand ranges from 2:3 to 1:1.

According to an embodiment of the present disclosure, the mass ratio of the phospholipid to the brain-targeting ligand is 5:6.

According to an embodiment of the present disclosure, the pharmaceutical composition further includes 5-fluorocytosine or an azole antifungal compound. Those skilled in the art can understand that when the composition exhibits a good fungicidal effect (e.g., against Cryptococcus) and improved safety, the pharmaceutical composition can be used in combination with substances conventionally known in the art to exhibit a good fungicidal effect on Cryptococcus can also exhibit a good fungicidal effect on Cryptococcus and improved safety.

According to an embodiment of the present disclosure, the pharmaceutical composition is in a form of an injection preparation or an oral preparation. In some preferred embodiments, when the pharmaceutical composition is in a form of an injection preparation or an oral preparation, the pharmaceutical composition can effectively exert a therapeutic effect.

According to an embodiment of the present disclosure, the pharmaceutical composition may further include a pharmaceutically acceptable carrier, including any solvent, solid excipient, diluent, binder, disintegrant, or other liquid excipient, dispersant, flavoring agent or suspending agent, surfactant, isotonic agent, thickener, emulsifier, preservative, solid binder, glidant or lubricant, among others, suitable for a specific target dosage form. Except to the extent that any conventional excipients are incompatible with the other ingredients in the pharmaceutical composition of the present disclosure, such as producing any adverse biological effects or interacting in a deleterious manner with any other components of the pharmaceutically acceptable composition, their use is also contemplated by the present disclosure.

For example, the pharmaceutical composition of the present disclosure can be incorporated into a medicament suitable for parenteral administration (e.g., intravenously, subcutaneously, intraperitoneally, intramuscularly). These medicaments can be prepared in a variety of forms. For example, liquid, semi-solid, and solid dosage forms, including but not limited to liquid solutions (e.g., injectable solutions and infusible solutions), dispersions or suspensions, tablets, pills, powders, liposomes, and suppositories. The medicament is typically in the form of an injectable solution, an infusible solution, or an oral preparation.

The effective amount of the pharmaceutical composition of the present disclosure may vary depending on the mode of administration and the severity of the disease to be treated. The selection of the preferred effective amount can be determined by a person skilled in the art based on various factors (e.g., through clinical trials). The factors include, but are not limited to: pharmacokinetic parameters of the active ingredient such as bioavailability, metabolism, half-life, etc.; and severity of the disease to be treated, weight of the patient, immune status of the patient, route of administration, etc. For example, several separate doses may be administered daily, or the dose may be proportionally reduced as required by the exigencies of the therapeutic situation.

In a second aspect of the present disclosure, the present disclosure provides a unit-dose preparation. According to an embodiment of the present disclosure, the unit-dose preparation includes an inositol phosphorylceramide synthase inhibitor and amphotericin B or a salt thereof. The unit-dose preparation includes 1 to 45 mg of amphotericin B or the salt thereof. As mentioned above, the inositol phosphorylceramide is an important and conserved component of fungal plasma membranes; however, when used alone, inositol phosphorylceramide synthase inhibitors, such as aureobasidin A, exhibit extremely low fungicidal activity against Cryptococcus. In the pharmaceutical composition according to an embodiment of the present disclosure, due to the presence of the inositol phosphorylceramide synthase inhibitor, the interaction between the inositol phosphorylceramide synthase inhibitor and amphotericin B not only enhances the inhibitory ability of the inositol phosphorylceramide synthase inhibitor on the synthesis of inositol phosphorylceramide in Cryptococcus, but also effectively prolongs the pharmacological action of amphotericin B or its salt in the body, leading to increased exposure levels in the body and higher contents of amphotericin B or its salt in brain tissue, thereby reducing the adverse reactions. As a result, both the efficacy and safety of amphotericin B or its salt are significantly improved, thereby effectively killing Cryptococcus and treating or preventing diseases caused by Cryptococcus, such as cryptococcal meningitis. In addition, it has been verified by experiments that the fungicidal effect and safety of the unit-dose preparation are superior to those of the currently used clinical combination of 5-fluorocytosine and amphotericin B.

It should be noted that the above-mentioned unit-dose preparation can be administered two, three, or four times per day, preferably twice daily. After conversion using this dose, it can effectively protect humans from, or alleviate, diseases such as cryptococcal meningitis. Those skilled in the art will appreciate that the specifications of the unit-dose preparation are not particularly limited. For example, the specifications of the unit-dose preparation may be set according to the characteristics of different target populations, for example, according to the weight of the target population, such as a weight of 40 kg, 50 kg, 60 kg, 70 kg, 80 kg, 90 kg, 100 kg, etc.; or according to the mass ratio of the inositol phosphorylceramide synthase inhibitor to amphotericin B or the salt thereof ranging from 1:3 to 5:1, specifically, 1:2, 1:1, 2:1, or 4:1, etc.; or the specifications of the unit-dose preparation may be set according to the characteristics of the target population and the mass ratio of the inositol phosphorylceramide synthase inhibitor to amphotericin B or the salt thereof.

According to an embodiment of the present disclosure, the unit-dose preparation may further include at least one of the following additional technical features.

According to an embodiment of the present disclosure, the inositol phosphorylceramide synthase inhibitor includes a cyclic depsipeptide antibiotic.

According to an embodiment of the present disclosure, the cyclic depsipeptide antibiotic includes at least one of aureobasidin A, enniatin, destruxin, emodepside, or fusafungine.

According to an embodiment of the present disclosure, the cyclic depsipeptide antibiotic includes aureobasidin A.

According to an embodiment of the present disclosure, the salt of amphotericin B includes at least one of: sulfate, phosphate, citrate, tartrate, fumarate, benzoate, adipate, succinate, mesylate, or maleate of amphotericin B.

According to an embodiment of the present disclosure, a mass ratio of the inositol phosphorylceramide synthase inhibitor to amphotericin B or the salt thereof ranges from 1:3 to 5:1.

According to an embodiment of the present disclosure, the mass ratio of the inositol phosphorylceramide synthase inhibitor to amphotericin B or the salt thereof ranges from 1:2 to 4:1.

According to an embodiment of the present disclosure, the mass ratio of the inositol phosphorylceramide synthase inhibitor to amphotericin B or the salt thereof is 1:2, 1:1, 2:1, or 4:1. According to some specific embodiments of the present disclosure, the mass ratio of the inositol phosphorylceramide synthase inhibitor to amphotericin B or the salt thereof has a certain influence on the efficacy and safety of the unit-dose preparation. When the mass ratio of the inositol phosphorylceramide synthase inhibitor to amphotericin B or the salt thereof is 1:2, 1:1, 2:1, or 4:1, the unit-dose preparation exhibits significantly enhanced fungicidal activity against Cryptococcus, further reduced adverse effects, and markedly improved efficacy and safety compared to the currently used clinical combination of amphotericin B and 5-flucytosine.

According to an embodiment of the present disclosure, the unit-dose preparation further includes a delivery carrier.

According to an embodiment of the present disclosure, the delivery carrier includes a liposome.

According to an embodiment of the present disclosure, the liposome includes a phospholipid.

According to an embodiment of the present disclosure, the phospholipid includes lecithin.

According to an embodiment of the present disclosure, the lecithin includes at least one of soybean lecithin or egg yolk lecithin.

According to an embodiment of the present disclosure, the liposome further includes a brain-targeting ligand.

According to an embodiment of the present disclosure, the brain-targeting ligand includes glutathione.

According to an embodiment of the present disclosure, a mass ratio of the phospholipid to the brain-targeting ligand ranges from 2:3 to 1:1.

According to an embodiment of the present disclosure, the mass ratio of the phospholipid to the brain-targeting ligand is 5:6.

According to an embodiment of the present disclosure, the unit-dose preparation further includes 5-fluorocytosine or an azole antifungal compound. Those skilled in the art can understand that when the existing ingredients of the unit-dose preparation, such as the inositol phosphorylceramide synthase inhibitor and amphotericin B or the salt thereof, exhibits a good fungicidal effect (e.g., against Cryptococcus) and improved safety, the unit-dose preparation can be used in combination with substances conventionally known in the art to exhibit a good fungicidal effect on Cryptococcus can also exhibit a good fungicidal effect on Cryptococcus and improved safety.

According to an embodiment of the present disclosure, the unit-dose preparation is in a form of an injection preparation or an oral preparation. The unit-dose preparation may be an oral preparation, an injection preparation, a pill, a sustained-release preparation, an implant, or an aerosol, etc., and is not particularly limited. In some preferred embodiments, when the unit-dose preparation is in a form of an injection preparation or an oral preparation, the unit-dose preparation can effectively exert a therapeutic effect.

In a third aspect of the present disclosure, the present disclosure provides a pharmaceutical combination. According to an embodiment of the present disclosure, the pharmaceutical combination includes: an inositol phosphorylceramide synthase inhibitor as a first active ingredient and amphotericin B or a salt thereof as a second active ingredient. As mentioned above, the inositol phosphorylceramide is an important and conserved component of fungal plasma membranes; however, when used alone, inositol phosphorylceramide synthase inhibitors, such as aureobasidin A, exhibit extremely low fungicidal activity against Cryptococcus. In the pharmaceutical combination according to an embodiment of the present disclosure, due to the presence of the inositol phosphorylceramide synthase inhibitor, the interaction between the inositol phosphorylceramide synthase inhibitor and amphotericin B not only enhances the inhibitory ability of the inositol phosphorylceramide synthase inhibitor on the synthesis of inositol phosphorylceramide in Cryptococcus, but also effectively prolongs the pharmacological action of amphotericin B or its salt in the body, leading to increased exposure levels in the body and higher contents of amphotericin B or its salt in brain tissue, thereby reducing the adverse reactions. As a result, both the efficacy and safety of amphotericin B or its salt are significantly improved, thereby effectively killing Cryptococcus and treating or preventing diseases caused by Cryptococcus, such as cryptococcal meningitis. In addition, it has been verified by experiments that the fungicidal effect and safety of the pharmaceutical combination are superior to those of the currently used clinical combination of 5-fluorocytosine and amphotericin B.

According to an embodiment of the present disclosure, the pharmaceutical combination may further include at least one of the following additional features.

According to an embodiment of the present disclosure, the inositol phosphorylceramide synthase inhibitor includes a cyclic depsipeptide antibiotic.

According to an embodiment of the present disclosure, the cyclic depsipeptide antibiotic includes at least one of aureobasidin A, enniatin, destruxin, emodepside, or fusafungine.

According to an embodiment of the present disclosure, the cyclic depsipeptide antibiotic includes aureobasidin A.

According to an embodiment of the present disclosure, the salt of amphotericin B includes at least one of: sulfate, phosphate, citrate, tartrate, fumarate, benzoate, adipate, succinate, mesylate, or maleate of amphotericin B.

According to an embodiment of the present disclosure, a mass ratio of the cyclic depsipeptide antibiotic to amphotericin B or the salt thereof ranges from 1:3 to 5:1.

According to an embodiment of the present disclosure, the mass ratio of the cyclic depsipeptide antibiotic to amphotericin B or the salt thereof ranges from 1:2 to 4:1.

According to an embodiment of the present disclosure, the mass ratio of the cyclic depsipeptide antibiotic to amphotericin B or the salt thereof is 1:2, 1:1, 2:1, or 4:1. According to some specific embodiments of the present disclosure, the mass ratio of the inositol phosphorylceramide synthase inhibitor to amphotericin B or the salt thereof has a certain influence on the efficacy and safety of the pharmaceutical combination. When the mass ratio of the inositol phosphorylceramide synthase inhibitor to amphotericin B or the salt thereof is 1:2, 1:1, 2:1, or 4:1, the pharmaceutical combination exhibits significantly enhanced fungicidal activity against Cryptococcus, further reduced adverse effects, and markedly improved efficacy and safety compared to the currently used clinical combination of amphotericin B and 5-flucytosine.

According to an embodiment of the present disclosure, the pharmaceutical combination further includes at least one of 5-fluorocytosine or an azole antifungal compound as a third active ingredient. Those skilled in the art can understand that when the existing ingredients in the pharmaceutical combination exhibits a good fungicidal effect and improved safety (e.g., the inositol phosphorylceramide synthetase inhibitor and amphotericin B or the salt thereof exhibits a good fungicidal effect and improved safety), the pharmaceutical combination can be used in combination with substances conventionally known in the art to exhibit a good fungicidal effect on Cryptococcus can also exhibit a good fungicidal effect on Cryptococcus and improved safety.

According to an embodiment of the present disclosure, the first active ingredient, the second active ingredient, and/or the third active ingredient are formulated collectively or separately.

According to an embodiment of the present disclosure, the first active ingredient, the second active ingredient, and/or the third active ingredient are encapsulated in one or more liposomes individually or collectively.

It should be noted that the pharmaceutical combination includes a combination separated in time and/or space, as long as the inositol phosphorylceramide synthase inhibitor and amphotericin B or the salt thereof can act collectively to achieve the purpose of the present disclosure. For example, the components contained in the pharmaceutical combination can be administered to a subject or a test sample as a whole, or can be administered to a subject or a test sample separately. When the components contained in the pharmaceutical combination are administered separately to a subject or a test sample, the individual components may be administered to the subject or the test sample simultaneously or sequentially.

According to an embodiment of the present disclosure, the liposome includes a phospholipid.

According to an embodiment of the present disclosure, the phospholipid includes lecithin.

According to an embodiment of the present disclosure, the lecithin includes at least one of soybean lecithin or egg yolk lecithin.

According to an embodiment of the present disclosure, the liposome further includes a brain-targeting ligand.

According to an embodiment of the present disclosure, the brain-targeting ligand includes glutathione.

According to an embodiment of the present disclosure, a mass ratio of the phospholipid to the brain-targeting ligand ranges from 2:3 to 1:1.

According to an embodiment of the present disclosure, the mass ratio of the phospholipid to the brain-targeting ligand is 5:6.

According to an embodiment of the present disclosure, the first active ingredient, the second active ingredient, and the third active ingredient are used simultaneously or separately. Those skilled in the art will appreciate that the first active ingredient, the second active ingredient, and the third active ingredient may be used individually or simultaneously. For example, the first active ingredient, the second active ingredient, and the third active ingredient may be used individually, or the first active ingredient and the second active ingredient may be used simultaneously, or the first active ingredient and the third active ingredient may be used simultaneously, or the second active ingredient and the third active ingredient may be used simultaneously, or the first active ingredient, the second active ingredient, and the third active ingredient may be used simultaneously.

According to an embodiment of the present disclosure, at least one of the first active ingredient, the second active ingredient, or the third active ingredient is in a form of an injection preparation or an oral preparation. At least one of the first active ingredient, the second active ingredient, or the third active ingredient may be an oral preparation, an injection preparation, a pill, a sustained-release preparation, an implant, or an aerosol, etc., and is not particularly limited. In some preferred embodiments, when at least one of the first active ingredient, the second active ingredient, or the third active ingredient is in a form of an injection preparation or an oral preparation, and each ingredient can effectively exert a therapeutic effect when at least one of the first active ingredient, the second active ingredient, or the third active ingredient are co-administrated.

In a fourth aspect of the present disclosure, the present disclosure provides a kit. According to an embodiment of the present disclosure, the kit includes: an inositol phosphorylceramide synthase inhibitor as a first active ingredient and amphotericin B or a salt thereof as a second active ingredient. As mentioned above, the inositol phosphorylceramide is an important and conserved component of fungal plasma membranes; however, when used alone, inositol phosphorylceramide synthase inhibitors, such as aureobasidin A, exhibit extremely low fungicidal activity against Cryptococcus. In the kit according to an embodiment of the present disclosure, due to the presence of the inositol phosphorylceramide synthase inhibitor, the interaction between the inositol phosphorylceramide synthase inhibitor and amphotericin B not only enhances the inhibitory ability of the inositol phosphorylceramide synthase inhibitor on the synthesis of inositol phosphorylceramide in Cryptococcus, but also effectively prolongs the pharmacological action of amphotericin B or its salt in the body, leading to increased exposure levels in the body and higher contents of amphotericin B or its salt in brain tissue, thereby reducing the adverse reactions. As a result, both the efficacy and safety of amphotericin B or its salt are significantly improved, thereby effectively killing Cryptococcus and treating or preventing diseases caused by Cryptococcus, such as cryptococcal meningitis. In addition, it has been verified by experiments that the fungicidal effect and safety of the kit are superior to those of the currently used clinical combination of 5-fluorocytosine and amphotericin B.

According to an embodiment of the present disclosure, the kit may further include at least one of the following additional features.

According to an embodiment of the present disclosure, the inositol phosphorylceramide synthase inhibitor includes a cyclic depsipeptide antibiotic.

According to an embodiment of the present disclosure, the cyclic depsipeptide antibiotic includes at least one of aureobasidin A, enniatin, destruxin, emodepside, or fusafungine.

According to an embodiment of the present disclosure, the cyclic depsipeptide antibiotic includes aureobasidin A.

According to an embodiment of the present disclosure, the salt of amphotericin B includes at least one of: sulfate, phosphate, citrate, tartrate, fumarate, benzoate, adipate, succinate, mesylate, or maleate of amphotericin B.

According to an embodiment of the present disclosure, a mass ratio of the cyclic depsipeptide antibiotic to amphotericin B or the salt thereof ranges from 1:3 to 5:1.

According to an embodiment of the present disclosure, the mass ratio of the cyclic depsipeptide antibiotic to amphotericin B or the salt thereof ranges from 1:2 to 4:1.

According to an embodiment of the present disclosure, the mass ratio of the cyclic depsipeptide antibiotic to amphotericin B or the salt thereof is 1:2, 1:1, 2:1, or 4:1. According to some specific embodiments of the present disclosure, the mass ratio of the inositol phosphorylceramide synthase inhibitor to amphotericin B or the salt thereof has a certain influence on the efficacy and safety of the kit. When the mass ratio of the inositol phosphorylceramide synthase inhibitor to amphotericin B or the salt thereof is 1:2, 1:1, 2:1, or 4:1, the kit exhibits significantly enhanced fungicidal activity against Cryptococcus, further reduced adverse effects, and markedly improved efficacy and safety compared to the currently used clinical combination of amphotericin B and 5-flucytosine.

According to an embodiment of the present disclosure, the kit further includes at least one of 5-fluorocytosine or an azole antifungal compound as a third active ingredient. Those skilled in the art can understand that when the existing ingredients of the kit, such as the inositol phosphorylceramide synthase inhibitor and amphotericin B or the salt thereof, exhibits a good fungicidal effect (e.g., against Cryptococcus) and improved safety, the kit can be used in combination with substances conventionally known in the art to exhibit a good fungicidal effect on Cryptococcus can also exhibit a good fungicidal effect on Cryptococcus and improved safety.

According to an embodiment of the present disclosure, the first active ingredient, the second active ingredient, and/or the third active ingredient are formulated collectively or separately.

According to an embodiment of the present disclosure, the first active ingredient, the second active ingredient, and/or the third active ingredient are encapsulated in one or more liposomes individually or collectively. According to some specific embodiments, the first active ingredient, the second active ingredient, and the third active ingredient can be encapsulated in multiple liposomes for use individually, or can be encapsulated in a single liposome for use simultaneously. For example, the first active ingredient, the second active ingredient, and the third active ingredient can be used individually, or the first active ingredient and the second active ingredient can be used simultaneously, or the first active ingredient and the third active ingredient can be used simultaneously, or the second active ingredient and the third active ingredient can be used simultaneously, or the first active ingredient, the second active ingredient, and the third active ingredient can be used simultaneously.

According to an embodiment of the present disclosure, the liposome includes a phospholipid.

According to an embodiment of the present disclosure, the phospholipid includes lecithin.

According to an embodiment of the present disclosure, the lecithin includes at least one of soybean lecithin or egg yolk lecithin.

According to an embodiment of the present disclosure, the liposome further includes a brain-targeting ligand.

According to an embodiment of the present disclosure, the brain-targeting ligand includes glutathione.

According to an embodiment of the present disclosure, a mass ratio of the phospholipid to the brain-targeting ligand ranges from 2:3 to 1:1.

According to an embodiment of the present disclosure, the mass ratio of the phospholipid to the brain-targeting ligand is 5:6.

According to an embodiment of the present disclosure, the first active ingredient, the second active ingredient, and the third active ingredient are used simultaneously or separately. It should be noted that the first active ingredient, the second active ingredient, and the third active ingredient include a combination separated in time and/or space, as long as the inositol phosphorylceramide synthase inhibitor and amphotericin B or the salt thereof can act collectively to achieve the purpose of the present disclosure. For example, each of the active components contained in the kit can be administered to a subject or a test sample as a whole, or can be administered to a subject or a test sample separately. When each of the active components contained in the kit is administered separately to a subject or a test sample, the individual components may be administered to the subject or the test sample simultaneously or sequentially.

According to an embodiment of the present disclosure, at least one of the first active ingredient, the second active ingredient, or the third active ingredient is in a form of an injection preparation or an oral preparation. According to an embodiment of the present disclosure, the medicament in the kit can be an oral preparation, an injection preparation, a pill, a sustained-release preparation, an implant, or an aerosol, etc., and is not particularly limited. In some preferred embodiments, when the medicament in the kit is in a form of an injection preparation or an oral preparation, the medicament in the kit can effectively exert a therapeutic effect.

In a fifth aspect of the present disclosure, the present disclosure provides a method for administering amphotericin B or a salt thereof. According to an embodiment of the present disclosure, the method includes co-administration of an inositol phosphorylceramide synthase inhibitor with amphotericin B or the salt thereof. As mentioned above, the inositol phosphorylceramide is an important and conserved component of fungal plasma membranes; however, when used alone, inositol phosphorylceramide synthase inhibitors, such as aureobasidin A, exhibit extremely low fungicidal activity against Cryptococcus. Amphotericin B is prone to causing adverse reactions when used alone, and has a short efficacy duration. In the method according to an embodiment of the present disclosure, due to the presence of the inositol phosphorylceramide synthase inhibitor, the interaction between the inositol phosphorylceramide synthase inhibitor and amphotericin B not only enhances the inhibitory ability of the inositol phosphorylceramide synthase inhibitor on the synthesis of inositol phosphorylceramide in Cryptococcus, but also effectively prolongs the pharmacological action of amphotericin B or its salt in the body, leading to increased exposure levels in the body and higher contents of amphotericin B or its salt in brain tissue, thereby reducing the adverse reactions. As a result, both the efficacy and safety of amphotericin B or its salt are significantly improved, thereby effectively killing Cryptococcus and treating or preventing diseases caused by Cryptococcus, such as cryptococcal meningitis. In addition, it has been verified by experiments that the fungicidal effect and safety of the method are superior to those of the currently used clinical combination of 5-fluorocytosine and amphotericin B.

According to an embodiment of the present disclosure, the above-mentioned method for administering amphotericin B or a salt thereof may further include at least one of the following additional technical features.

According to an embodiment of the present disclosure, the co-administration includes simultaneous or separate administration of the inositol phosphorylceramide synthase inhibitor and amphotericin B or the salt thereof.

According to an embodiment of the present disclosure, the inositol phosphorylceramide synthase inhibitor includes a cyclic depsipeptide antibiotic.

According to an embodiment of the present disclosure, the cyclic depsipeptide antibiotic includes at least one of aureobasidin A, enniatin, destruxin, emodepside, or fusafungine.

According to an embodiment of the present disclosure, the cyclic depsipeptide antibiotic includes aureobasidin A.

According to an embodiment of the present disclosure, the salt of amphotericin B includes at least one of: sulfate, phosphate, citrate, tartrate, fumarate, benzoate, adipate, succinate, mesylate, or maleate of amphotericin B.

According to an embodiment of the present disclosure, the cyclic depsipeptide antibiotic and amphotericin B or the salt thereof are co-administered at a mass ratio ranging from 1:3 to 5:1.

According to an embodiment of the present disclosure, the cyclic depsipeptide antibiotic and amphotericin B or the salt thereof are co-administered at a mass ratio ranging from 1:2 to 4:1.

According to an embodiment of the present disclosure, the cyclic depsipeptide antibiotic and amphotericin B or the salt thereof are co-administered at a mass ratio of 1:2, 1:

1, 2:1, or 4:1.

According to an embodiment of the present disclosure, the co-administration further includes administering at least one of 5-fluorocytosine or an azole antifungal compound.

According to an embodiment of the present disclosure, the at least one of 5-fluorocytosine or the azole antifungal compound, the cyclic depsipeptide antibiotic, and amphotericin B or the salt thereof are administered simultaneously or separately.

According to an embodiment of the present disclosure, at least one of the at least one of 5-fluorocytosine or the azole antifungal compound, the cyclic depsipeptide antibiotic, or amphotericin B or the salt thereof is in a form of an injection preparation or an oral preparation.

In a sixth aspect of the present disclosure, the present disclosure provides a method for prolonging half-life of amphotericin B or a salt thereof. According to an embodiment of the present disclosure, the method includes co-administration of an inositol phosphorylceramide synthase inhibitor with amphotericin B or the salt thereof. As mentioned above, amphotericin B is prone to causing adverse reactions when used alone, and has a short efficacy duration. In the method according to an embodiment of the present disclosure, due to the presence of the inositol phosphorylceramide synthase inhibitor, the interaction between the inositol phosphorylceramide synthase inhibitor and amphotericin B effectively prolongs the pharmacological action of amphotericin B or its salt in the body and prolongs its half-time, leading to increased exposure levels in the body and higher contents of amphotericin B or its salt in brain tissue, thereby reducing the adverse reactions. As a result, both the efficacy and safety of amphotericin B or its salt are significantly improved.

According to an embodiment of the present disclosure, the method for prolonging the half-life of amphotericin B or a salt thereof may further include at least one of the following additional technical features.

According to an embodiment of the present disclosure, the co-administration includes simultaneous or separate administration of the inositol phosphorylceramide synthase inhibitor and amphotericin B or the salt thereof.

According to an embodiment of the present disclosure, the inositol phosphorylceramide synthase inhibitor includes a cyclic depsipeptide antibiotic.

According to an embodiment of the present disclosure, the cyclic depsipeptide antibiotic includes at least one of aureobasidin A, enniatin, destruxin, emodepside, or fusafungine.

According to an embodiment of the present disclosure, the cyclic depsipeptide antibiotic includes aureobasidin A.

According to an embodiment of the present disclosure, the salt of amphotericin B includes at least one of: sulfate, phosphate, citrate, tartrate, fumarate, benzoate, adipate, succinate, mesylate, or maleate of amphotericin B.

According to an embodiment of the present disclosure, the cyclic depsipeptide antibiotic and amphotericin B or the salt thereof are co-administered at a mass ratio ranging from 1:3 to 5:1.

According to an embodiment of the present disclosure, the cyclic depsipeptide antibiotic and amphotericin B or the salt thereof are co-administered at a mass ratio ranging from 1:2 to 4:1.

According to an embodiment of the present disclosure, the cyclic depsipeptide antibiotic and amphotericin B or the salt thereof are co-administered at a mass ratio of 1:2, 1:1, 2:1, or 4:1.

According to an embodiment of the present disclosure, the co-administration further includes administering at least one of 5-fluorocytosine or an azole antifungal compound.

According to an embodiment of the present disclosure, the at least one of 5-fluorocytosine or the azole antifungal compound, the cyclic depsipeptide antibiotic, and amphotericin B or the salt thereof are administered simultaneously or separately.

According to an embodiment of the present disclosure, at least one of the at least one of 5-fluorocytosine or the azole antifungal compound, the cyclic depsipeptide antibiotic, or amphotericin B or the salt thereof is in a form of an injection preparation or an oral preparation.

In a seventh aspect of the present disclosure, the present disclosure provides a method for improving an inhibitory activity of amphotericin B or a salt thereof against Cryptococcus. According to an embodiment of the present disclosure, the method includes co-administration of an inositol phosphorylceramide synthase inhibitor with amphotericin B or the salt thereof. As mentioned above, amphotericin B is prone to causing adverse reactions when used alone, and has a short efficacy duration. According to the method of the embodiment of the present disclosure, the interaction between the inositol phosphorylceramide synthase inhibitor and amphotericin B significantly enhances the inhibitory ability of amphotericin B or the salt thereof against Cryptococcus. As a result, under the same dosage of amphotericin B, the combination of the inositol phosphorylceramide synthase inhibitor and amphotericin B or its salt provides superior antifungal efficacy and improved safety compared to the combination of amphotericin B and 5-flucytosine.

It should be noted that the “against Cryptococcus” is not particularly limited, and at least includes inhibiting at least one of the proliferation activity, differentiation activity, or growth and metabolic activity of Cryptococcus.

According to an embodiment of the present disclosure, the above-mentioned method for improving the inhibitory activity of amphotericin B or a salt thereof against Cryptococcus may further include at least one of the following additional technical features.

According to an embodiment of the present disclosure, the co-administration includes simultaneous or separate administration of the inositol phosphorylceramide synthase inhibitor and amphotericin B or the salt thereof.

According to an embodiment of the present disclosure, the inositol phosphorylceramide synthase inhibitor includes a cyclic depsipeptide antibiotic.

According to an embodiment of the present disclosure, the cyclic depsipeptide antibiotic includes at least one of aureobasidin A, enniatin, destruxin, emodepside, or fusafungine.

According to an embodiment of the present disclosure, the cyclic depsipeptide antibiotic includes aureobasidin A.

According to an embodiment of the present disclosure, the salt of amphotericin B includes at least one of: sulfate, phosphate, citrate, tartrate, fumarate, benzoate, adipate, succinate, mesylate, or maleate of amphotericin B.

According to an embodiment of the present disclosure, the cyclic depsipeptide antibiotic and amphotericin B or the salt thereof are co-administered at a mass ratio ranging from 1:3 to 5:1.

According to an embodiment of the present disclosure, the cyclic depsipeptide antibiotic and amphotericin B or the salt thereof are co-administered at a mass ratio ranging from 1:2 to 4:1.

According to an embodiment of the present disclosure, the cyclic depsipeptide antibiotic and amphotericin B or the salt thereof are co-administered at a mass ratio of 1:2, 1:1, 2:1, or 4:1.

According to an embodiment of the present disclosure, the co-administration further includes administering at least one of 5-fluorocytosine or an azole antifungal compound.

According to an embodiment of the present disclosure, the at least one of 5-fluorocytosine or the azole antifungal compound, the cyclic depsipeptide antibiotic, and amphotericin B or the salt thereof are administered simultaneously or separately.

According to an embodiment of the present disclosure, at least one of the at least one of 5-fluorocytosine or the azole antifungal compound, the cyclic depsipeptide antibiotic, or amphotericin B or the salt thereof is in a form of an injection preparation or an oral preparation.

In an eighth aspect of the present disclosure, the present disclosure provides a method for improving an inhibitory activity of an inositol phosphorylceramide synthetase inhibitor against Cryptococcus neoformans. According to an embodiment of the present disclosure, the method includes co-administration of the inositol phosphorylceramide synthetase inhibitor with amphotericin B or a salt thereof. As mentioned above, the inositol phosphorylceramide is an important and conserved component of fungal plasma membranes; however, when used alone, inositol phosphorylceramide synthase inhibitors, such as aureobasidin A, exhibit extremely low fungicidal activity against Cryptococcus. In the method according to an embodiment of the present disclosure, the interaction between the inositol phosphorylceramide synthase inhibitor and amphotericin B significantly enhances the inhibitory ability of the inositol phosphorylceramide synthase inhibitor on the synthesis of inositol phosphorylceramide in Cryptococcus. As a result, under the same dosage of amphotericin B, the combination of the inositol phosphorylceramide synthase inhibitor and amphotericin B or its salt provides superior antifungal efficacy and improved safety compared to the combination of amphotericin B and 5-flucytosine.

It should be noted that the “against Cryptococcus” is not particularly limited, and at least includes inhibiting at least one of the proliferation activity, differentiation activity, or growth and metabolic activity of Cryptococcus.

According to an embodiment of the present disclosure, the above-mentioned method for improving the inhibitory activity of the inositol phosphorylceramide synthetase inhibitor against Cryptococcus neoformans may further include at least one of the following additional technical features.

According to an embodiment of the present disclosure, the co-administration includes simultaneous or separate administration of the inositol phosphorylceramide synthase inhibitor and amphotericin B or the salt thereof.

According to an embodiment of the present disclosure, the inositol phosphorylceramide synthase inhibitor includes a cyclic depsipeptide antibiotic.

According to an embodiment of the present disclosure, the cyclic depsipeptide antibiotic includes at least one of aureobasidin A, enniatin, destruxin, emodepside, or fusafungine.

According to an embodiment of the present disclosure, the cyclic depsipeptide antibiotic includes aureobasidin A.

According to an embodiment of the present disclosure, the salt of amphotericin B includes at least one of: sulfate, phosphate, citrate, tartrate, fumarate, benzoate, adipate, succinate, mesylate, or maleate of amphotericin B.

According to an embodiment of the present disclosure, the cyclic depsipeptide antibiotic and amphotericin B or the salt thereof are co-administered at a mass ratio ranging from 1:3 to 5:1.

According to an embodiment of the present disclosure, the cyclic depsipeptide antibiotic and amphotericin B or the salt thereof are co-administered at a mass ratio ranging from 1:2 to 4:1.

According to an embodiment of the present disclosure, the cyclic depsipeptide antibiotic and amphotericin B or the salt thereof are co-administered at a mass ratio of 1:2, 1:1, 2:1, or 4:1.

According to an embodiment of the present disclosure, the co-administration further includes administering at least one of 5-fluorocytosine or an azole antifungal compound.

According to an embodiment of the present disclosure, the at least one of 5-fluorocytosine or the azole antifungal compound, the cyclic depsipeptide antibiotic, and amphotericin B or the salt thereof are administered simultaneously or separately.

According to an embodiment of the present disclosure, at least one of the at least one of 5-fluorocytosine or the azole antifungal compound, the cyclic depsipeptide antibiotic, or amphotericin B or the salt thereof is in a form of an injection preparation or an oral preparation.

In a ninth aspect of the present disclosure, the present disclosure provides use of the above-mentioned pharmaceutical composition, unit-dose preparation, pharmaceutical combination, or kit in the manufacture of a medicament for treating or preventing a disease associated with Cryptococcus infection. As mentioned above, the pharmaceutical composition, unit-dose preparation, pharmaceutical combination, or kit can effectively kill Cryptococcus, Candida albicans, Candida parapsilosis, and Rhizopus arrhizus, among others. In particular, they exhibit superior therapeutic efficacy in the treatment or prevention of diseases caused by Cryptococcus. It has been verified by experiments that the fungicidal effect and safety of the pharmaceutical composition, unit-dose preparation, pharmaceutical combination, or kit are superior to those of the currently used clinical combination of 5-fluorocytosine and amphotericin B. Therefore, the medicament containing the above substances also has the above effects.

According to an embodiment of the present disclosure, the disease associated with Cryptococcus infection includes cryptococcal meningitis. According to some specific embodiments of the present disclosure, the above-mentioned medicament exhibits high safety and high killing ability against cryptococcal meningitis.

In a tenth aspect of the present disclosure, the present disclosure provides use of the above-mentioned pharmaceutical composition, unit-dose preparation, pharmaceutical combination, or kit in treating or preventing a disease associated with Cryptococcus infection. As mentioned above, the pharmaceutical composition, unit-dose preparation, pharmaceutical combination, or kit can effectively kill Cryptococcus, Candida albicans, Candida parapsilosis, and Rhizopus arrhizus, among others. In particular, they exhibit superior therapeutic efficacy in the treatment or prevention of diseases caused by Cryptococcus. It has been verified by experiments that the fungicidal effect and safety of the pharmaceutical composition, unit-dose preparation, pharmaceutical combination, or kit are superior to those of the currently used clinical combination of 5-fluorocytosine and amphotericin B. Therefore, the medicament containing the above substances also has the above effects.

According to an embodiment of the present disclosure, the disease associated with Cryptococcus infection includes cryptococcal meningitis. According to some specific embodiments of the present disclosure, the above-mentioned medicament exhibits high safety and high killing ability against cryptococcal meningitis.

In an eleventh aspect of the present disclosure, the present disclosure provides the above-mentioned pharmaceutical composition, unit-dose preparation, pharmaceutical combination, or kit for use in treating or preventing a disease associated with Cryptococcus infection. As mentioned above, the pharmaceutical composition, unit-dose preparation, pharmaceutical combination, or kit can effectively kill Cryptococcus, Candida albicans, Candida parapsilosis, and Rhizopus arrhizus, among others. In particular, they exhibit superior therapeutic efficacy in the treatment or prevention of diseases caused by Cryptococcus. It has been verified by experiments that the fungicidal effect and safety of the pharmaceutical composition, unit-dose preparation, pharmaceutical combination, or kit are superior to those of the currently used clinical combination of 5-fluorocytosine and amphotericin B. Therefore, the medicament containing the above substances also has the above effects.

According to an embodiment of the present disclosure, the disease associated with Cryptococcus infection includes cryptococcal meningitis. According to some specific embodiments of the present disclosure, the above-mentioned medicament exhibits high safety and high killing ability against cryptococcal meningitis.

In a twelfth aspect of the present disclosure, the present disclosure provides a method for treating or preventing a disease associated with Cryptococcus infection. According to an embodiment of the present disclosure, the method includes: administering to a subject a pharmaceutically acceptable amount of the above-mentioned pharmaceutical composition, the above-mentioned unit-dose preparation, or the above-mentioned pharmaceutical combination or kit. As mentioned above, the pharmaceutical composition, unit-dose preparation, pharmaceutical combination, or kit can effectively kill Cryptococcus, Candida albicans, Candida parapsilosis, and Rhizopus arrhizus, among others. In particular, they exhibit superior therapeutic efficacy in the treatment or prevention of diseases caused by Cryptococcus. It has been verified by experiments that the fungicidal effect and safety of the pharmaceutical composition, unit-dose preparation, pharmaceutical combination, or kit are superior to those of the currently used clinical combination of 5-fluorocytosine and amphotericin B. Therefore, the method of the present disclosure can effectively treat or prevent a disease associated with Cryptococcus infection.

According to an embodiment of the present disclosure, the disease associated with Cryptococcus infection includes cryptococcal meningitis. According to some specific embodiments of the present disclosure, the above-mentioned medicament has high safety and high killing ability against cryptococcal meningitis.

### BRIEF DESCRIPTION OF THE DRAWINGS

FIG. 1 is a graph showing the test results of the effect of AbA and IPC on the fungicidal effect of AmB according to Example 1 of the present disclosure.

FIG. 2 is a graph showing the test results of the FICI values of the combination of AbA and AmB versus the combination of 5-FC and AmB according to Example 1 of the present disclosure.

FIG. 3 is a graph showing the test results of the in vitro fungicidal activity of different concentrations of the combination of AbA and AmB against Cryptococcus according to Example 2 of the present disclosure.

FIG. 4 is a schematic diagram illustrating the experimental procedure for treating a mouse model of Cryptococcus neoformans brain infection with various doses of AmB, AbA, AmB liposomes, AbA liposomes, and AmB:AbA co-encapsulated liposomes according to Example 3 of the present disclosure, wherein “Brain infection model” indicates the Cryptococcus neoformans brain infection model, “Drug treatment” refers to the treatment regimen, the drugs used (labeled as “drug”) include AmB-lip (AmB liposomes), ABA-lip (AbA liposomes), and ABA:AmB-lip (AbA:AmB co-encapsulated liposomes), and “Sacrifice time” indicates the time of sacrifice.

FIG. 5A is a graph showing the test results of the fungal burden in the brains of mice infected with Cryptococcus neoformans after treatment with various doses of AmB, AbA, AmB liposomes, AbA liposomes, or AbA:AmB co-encapsulated liposomes according to Example 3 of the present disclosure.

FIG. 5B is a graph showing the test results of the survival of mice infected with Cryptococcus neoformans after treatment with various doses of AmB, AbA, AmB liposomes, AbA liposomes, or AbA:AmB co-encapsulated liposomes according to Example 3 of the present disclosure.

### DETAILED DESCRIPTION

Embodiments of the present disclosure are described in detail below, examples of which are shown in the accompanying drawings, wherein the same or similar reference numerals throughout represent the same or similar elements or elements having the same or similar functions. The embodiments described below with reference to the accompanying drawings are exemplary and are intended to be used to illustrate the present disclosure, but should not be construed as limiting the present disclosure.

As used herein, the terms “include” or “comprise” are open expressions, that is, including the contents specified in the present disclosure but not excluding other contents.

As used herein, the terms “optionally” or “optional” generally mean that the subsequently described event or circumstance may but need not occur, and that the description includes instances where the event or circumstance occurs and instances where it does not.

In the process of describing the present disclosure, the relevant terms as used herein are explained and illustrated. These explanations and illustrations are only for the convenience of understanding of the scheme and cannot be regarded as limitations on the protection scheme of the present disclosure.

As used herein, amphotericin B has a molecular formula of C₄₇H₇₃NO₁₇, and a chemical structure of:

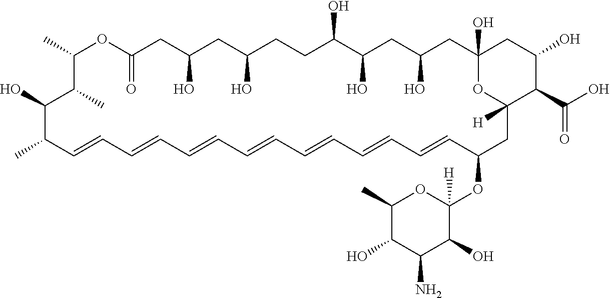 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)C[C@@H](O)[C@H](O)CC[C@@H](O)C[C@@H](O)CC(=O)O[C@H]1C)C[C@H](O)[C@H]2C(=O)O]

As used herein, aureobasidin A (AbA) has a molecular formula of C₆₀H₉₂N₈O₁₁, and a chemical structure of:

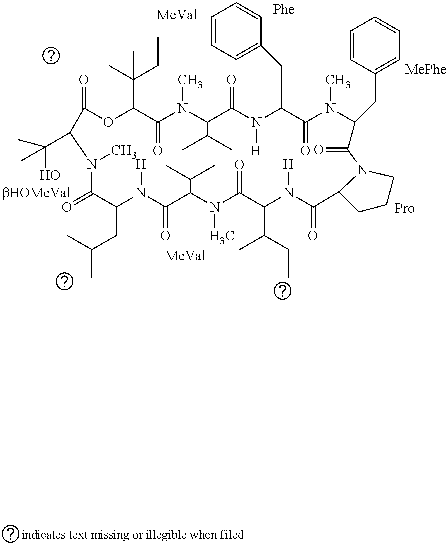 [SMILES: CCC(C)C1NC(=O)C2CCCN2C(=O)C(Cc2ccccc2)N(C)C(=O)C(Cc2ccccc2)NC(=O)C(C(C)C)N(C)C(=O)C(C(C)(C)CC)OC(=O)C(C(C)(C)O)N(C)C(=O)C(CC(C)C)NC(=O)C(C(C)C)N(C)C1=O]

As used herein, the “unit-dose preparation” refers to a dosage form designed to be administered in a single dose per administration.

As used herein, the term “pharmaceutical combination” refers to the simultaneous or sequential use of two or more medicaments to achieve a therapeutic purpose, the result of which is mainly to increase the efficacy of the medicaments or to reduce the toxic or side effects of the medicaments.

As used herein, the “kit” refers to a box containing a set of one or more medicaments. The kit provides the medicaments in predetermined ratios, which allows each medicament to be stored and packaged in proportions and form that exerts a better efficacy and facilitate convenient and effective use of each medicament, wherein one or more medicaments can be packaged separately or collectively.

As used herein, the term “pharmaceutically acceptable excipient” includes any solvent, dispersion medium, coating material, surfactant, antioxidant, preservative (e.g., antibacterial agent, antifungal agent), isotonic agent, salt, medicament stabilizer, binder, excipient, dispersant, lubricant, or a combination thereof, which are known to those skilled in the art (e.g., Remington's Pharmaceutical Sciences, 18th Ed. Mack Printing Company, 1990, pp. 1289-1329). Except insofar as any conventional carrier is incompatible with the active ingredient, their use in the therapeutic or pharmaceutical compositions is contemplated.

As used herein, the term “administering” or “administration” refers to introducing a predetermined amount of a substance into a patient by some suitable means. The pharmaceutical composition and unit-dose preparation of the present disclosure can be administered via any common route as long as it can reach the intended tissue. Various modes of administration are contemplated, including peritoneal, intravenous, intramuscular, subcutaneous, etc., but the present disclosure is not limited to these exemplified modes of administration. Preferably, the composition of the present disclosure is administered by intravenous injection or subcutaneous injection.

As used herein, the term “treatment” refers to the use of medicaments to obtain a desired pharmacological and/or physiological effect. The effect may be prophylactic, in terms of complete or partial prevention of the disease or its symptoms, and/or therapeutic, in terms of partial or complete cure of the disease and/or adverse effects resulting from the disease. As used herein, “treatment” covers diseases in mammals, particularly humans, and includes: (a) preventing the occurrence of a disease or condition in an individual who is susceptible to the disease but has not yet been diagnosed with the disease; (b) inhibiting the disease, such as arresting the progression of the disease; or (c) alleviating the disease, such as alleviating the symptoms associated with the disease. As used herein, the “treatment” encompasses any administration of a pharmaceutical composition or unit-dose preparation to an individual to treat, cure, alleviate, ameliorate, mitigate, or inhibit a disease in the individual, including but not limited to administering the pharmaceutical composition or unit-dose preparation of the present disclosure to an individual in need thereof.

The scheme of the present disclosure will be explained below with reference to embodiments. Those skilled in the art will appreciate that the following embodiments are only used to illustrate the present disclosure and should not be considered to limit the scope of the present disclosure. Where specific techniques or conditions are not specified in the examples, they are performed according to techniques or conditions described in the literature in the art or according to the product description. The reagents or instruments used are conventional products that can be obtained commercially without indicating the manufacturer.

### Example 1: Evaluation of the Antifungal Activity of Combined Use of AbA and AmB

In this example, the antifungal activity of AbA was evaluated. Specifically, the inventors tested the activity of AbA alone and in combination with AmB against Cryptococcus. In addition, the inventors also tested the effect of purified IPC on the antifungal activity of AbA alone and in combination with AmB against Cryptococcus. The specific experimental procedures are as follows.

In this example, a total of 5 groups were set up, including AbA alone, AmB alone, AbA combined with AmB, AbA combined with AmB in the presence of 1 mg/mL IPC, and AbA combined with AmB in the presence of 10 mg/mL IPC. The fungicidal efficacy of the above 5 groups of treatments against Cryptococcus neoformans var. grubii H99 (a laboratory strain) was evaluated. The survival rate of Cryptococcus after 1 hour of treatment with AmB in YPD medium was determined by calculating the CFU ratio between AmB-treated and untreated samples. AbA and IPC were added 6 hours prior to AmB treatment as a pre-treatment. AbA was purchased from Biorigin, AmB from Inalco, and IPC was extracted from an ERG6 gene knockout H99 mutant strain defective in ergosterol biosynthesis.

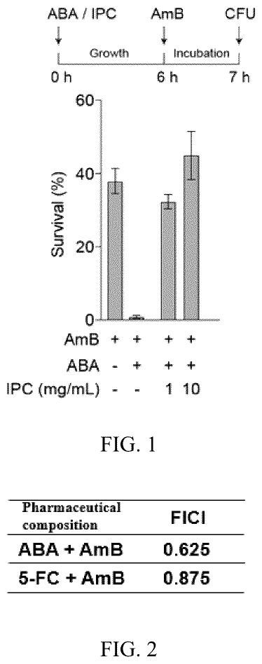

The results in FIG. 1 show that AbA (1 μg/mL) alone exhibited no fungicidal effect against Cryptococcus. However, when combined with AmB (1 μg/mL), AbA significantly enhanced the fungicidal effect of AmB, reducing the survival rate of Cryptococcus neoformans from 39% to less than 1%. Furthermore, the addition of purified IPC at final concentrations of 1 mg/mL and 10 mg/mL restored the fungicidal effect of the AbA-AmB combination to that of AmB alone, indicating that IPC can significantly affect the fungicidal efficiency of AbA and AmB.

The results in FIG. 2 show that the fractional inhibitory concentration index (FICI) of the AbA-AmB combination reached 0.625, indicating a clear additive effect. In comparison, the clinically used combination of flucytosine and AmB showed an FICI of 0.875. Therefore, the AbA-AmB combination demonstrates superior efficacy relative to the flucytosine-AmB combination.

### Example 2: In Vitro Fungicidal Activity of Combined Use of AbA and AmB

As shown in Table 1, a total of 6 groups were set up in this example to evaluate the fungicidal effect of different treatments against Cryptococcus neoformans. The experimental procedures were performed with reference to Example 1. Specifically, Cryptococcus neoformans cells were pretreated with various concentrations of AbA for 6 hours, followed by treatment with 0.5 μg/mL or 1 μg/mL of AmB. The survival rate was determined by calculating the ratio of colony-forming units (CFUs) between the AmB-treated and untreated samples.

TABLE 1

| | Group A | Group B | Group C | Group D | Group E | Group F |
|---|---|---|---|---|---|---|
| Item | (μg/mL) | (μg/mL) | (μg/mL) | (μg/mL) | (μg/mL) | (μg/mL) |
| AbA | 0 | 0.5 | 1.0 | 0 | 0.5 | 1.0 |
| AmB | 0.5 | 0.5 | 0.5 | 1.0 | 1.0 | 1.0 |

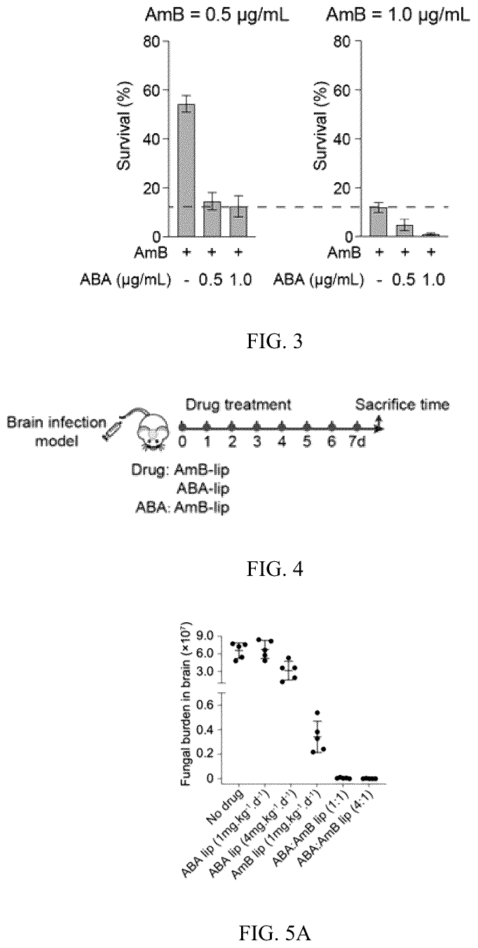

The results in FIG. 3 show that the addition of 0.5 μg/mL and 1 μg/mL of AbA allowed AmB at a concentration of 0.5 μg/mL to achieve a fungicidal efficiency equivalent to that of 1 μg/mL AmB alone. This will reduce the clinically required dose of AmB and minimize its side effects.

### Example 3: In Vivo Fungicidal Activity of Combined Use of AbA and AmB

This example was conducted based on the results of Example 1 and Example 2. Specifically, the fungicidal effects of the prepared AbA and AmB liposomes were evaluated using a mouse model of Cryptococcus neoformans infection. The specific experimental procedures are as follows.

### 3.1: Preparation of AmB Liposomes, AbA Liposomes, and AmB:AbA (or AbA:AmB) Co-Encapsulated Liposomes

The AmB:AbA co-encapsulated liposomes were prepared by the inventors using the thin film hydration method. Specifically, 1.2 mg of glutathione and 10 mg of egg yolk lecithin were dissolved in a methanol:chloroform (v:v=1:1) mixed solution. The organic solvents were removed by rotary evaporation in a round-bottom flask to form a film. 1 mg AmB and 1 mg AbA were dissolved in 50 μL DMSO. 10 μL AmB and 10 μL, 20 μL, or 40 μL AbA were added to glucose injection solution to allow the mass ratio of AmB to AbA to be 1:1, 1:2, and 1:4, respectively. The mixture was added into a round-bottom flask and hydrated at 37° C. with shaking, and then ultrasonicated with a 600W probe-type sonicator until a translucent solution was obtained, yielding unilamellar liposomes. In addition, the inventors also prepared AmB liposomes and AbA liposomes separately in a similar manner.

### 3.2: In Vivo Fungicidal Activity Evaluation

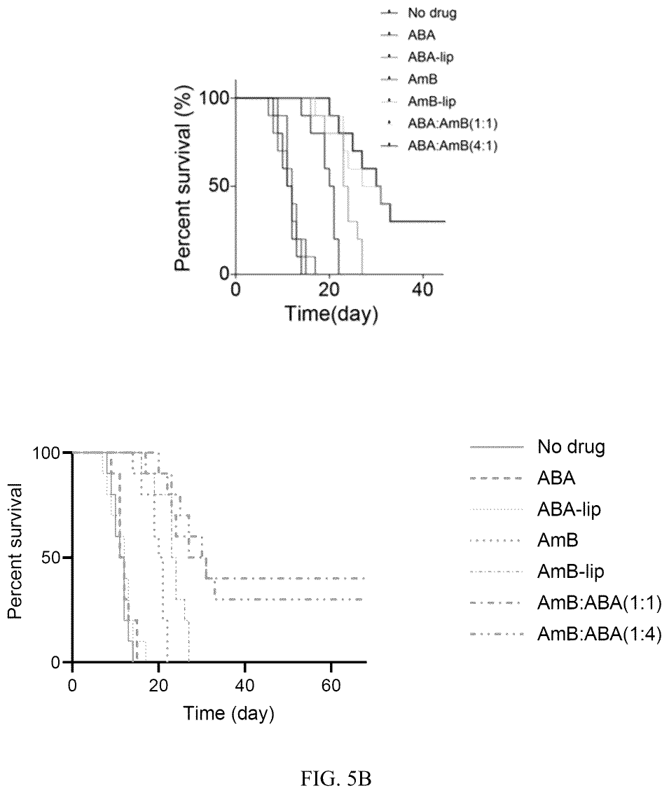

In this example, a mouse model of Cryptococcus neoformans brain infection was established by tail vein injection of Cryptococcus neoformans clinical strain H99 into C57BL/6J female mice. The mouse model of Cryptococcus neoformans brain infection was established specifically as follows. The concentration of Cryptococcus was adjusted to 1×10⁷ CFU/mL, and 50 μL of cells (5×10⁵ CFU/mouse) was injected into restrained 8- to 10-week-old C57BL/6J female mice via the tail vein. The successfully constructed mouse model of Cryptococcus neoformans brain infection was administrated AmB:AbA co-encapsulated liposomes, AmB liposomes, AbA liposomes, AmB, AbA, and PBS (no drug) every day for 7 consecutive days. The dosage was 1 mg kg⁻¹ d⁻¹ for AmB and 4 mg kg⁻¹ d⁻¹ for AbA. The death of mice was recorded, and 5 mice from each group were sacrificed on the eighth day to determine the fungal burden in the brain. The specific experimental process settings were shown in FIG. 4. The results were shown in FIG. 5A that, compared with AmB liposomes, AmB:AbA co-encapsulated liposomes effectively eliminated Cryptococcus in the brain and reduced the fungal burden in the brain by two orders of magnitude. AbA liposomes alone exhibited no effect in eliminating Cryptococcus in the brain. Furthermore, the results of FIG. 5B showed that 4 (4/10) mice treated with AmB:AbA co-encapsulated liposomes survived to the end of the experiment (more than two months) after cessation of treatment, while all mice treated with AmB liposomes alone died of recurrent infection within 20 days after treatment withdrawal. Therefore, AmB:AbA co-encapsulated liposomes demonstrated superior antifungal efficacy compared to AmB liposomes alone.

In the specification, the description of the reference terms such as “one embodiment”, “some embodiments”, “example”, “specific example”, or “some examples” means that the specific features, structures, materials, or characteristics described with reference to the embodiment or example are included in at least an embodiment or example of the present disclosure. In this specification, exemplary descriptions of the foregoing terms do not necessarily refer to the same embodiment or example. Moreover, the specific features, structures, materials, or characteristics described may be combined in any one or more embodiments or examples in a suitable manner. Furthermore, those skilled in the art may combine different embodiments or examples and features of different embodiments or examples described in this specification, unless they are contradictory to each other. Although embodiments of the present disclosure are illustrated and described above, it can be understood that the above embodiments are illustrative and should not be construed as limitations of the present disclosure. Those skilled in the art can make changes, modifications, substitutions, and variations to the above embodiments within the scope of the present disclosure.

What is claimed is:

1. A pharmaceutical composition, comprising:
an inositol phosphorylceramide synthase inhibitor; and
amphotericin B or a salt thereof.

2. The pharmaceutical composition according to claim 1, provided as a unit-dose preparation, the pharmaceutical composition comprising:
an inositol phosphorylceramide synthase inhibitor; and
1 to 45 mg of amphotericin B or a salt thereof.

3. The pharmaceutical composition according to claim 1, provided as a pharmaceutical combination or kit, the pharmaceutical composition comprising:
an inositol phosphorylceramide synthase inhibitor, as a first active ingredient; and
amphotericin B or a salt thereof, as a second active ingredient.

4. The pharmaceutical composition according to claim 1, wherein:
the inositol phosphorylceramide synthase inhibitor comprises a cyclic depsipeptide antibiotic; or
the salt of amphotericin B, if present, comprises at least one of: sulfate, phosphate, citrate, tartrate, fumarate, benzoate, adipate, succinate, mesylate, deoxycholate, or maleate of amphotericin B;
optionally, a mass ratio of the inositol phosphorylceramide synthase inhibitor to amphotericin B or the salt thereof ranges from 1:3 to 5:1.

5. The pharmaceutical composition according to claim 4, wherein the cyclic depsipeptide antibiotic comprises at least one of aureobasidin A, enniatin, destruxin, emodepside, or fusafungine.

6. The pharmaceutical composition according to claim 4, wherein:
the cyclic depsipeptide antibiotic comprises aureobasidin A;
optionally, the mass ratio of the inositol phosphorylceramide synthase inhibitor to amphotericin B or the salt thereof ranges from 1:2 to 4:1.

7. The pharmaceutical composition according to claim 4, wherein the mass ratio of the inositol phosphorylceramide synthase inhibitor to amphotericin B or the salt thereof is 1:2, 1:
1, 2:1, or 4:1.

8. The pharmaceutical composition according to claim 4, further comprising a delivery carrier;
optionally, the delivery carrier comprises a liposome.

9. The pharmaceutical composition according to claim 4, comprising:
an inositol phosphorylceramide synthase inhibitor, as a first active ingredient;
amphotericin B or a salt thereof, as a second active ingredient; and
at least one of 5-fluorocytosine or an azole antifungal compound, as a third active ingredient;
optionally, wherein the first active ingredient, the second active ingredient, and/or the third active ingredient are formulated collectively or separately;
optionally, wherein the first active ingredient, the second active ingredient, and/or the third active ingredient are encapsulated in one or more liposomes individually or collectively.

10. The pharmaceutical composition according to claim 8, wherein the liposome comprises a phospholipid.

11. The pharmaceutical composition according to claim 10, wherein the liposome further comprises a brain-targeting ligand; optionally, the brain-targeting ligand comprises glutathione.

12. The pharmaceutical composition according to claim 11, wherein the phospholipid comprises lecithin;
optionally, wherein the lecithin comprises at least one of soybean lecithin or egg yolk lecithin;
optionally, wherein a mass ratio of the phospholipid to the brain-targeting ligand ranges from 2:3 to 1:1.

13. The pharmaceutical composition according to claim 12, wherein the mass ratio of the phospholipid to the brain-targeting ligand is 5:6.

14. The pharmaceutical composition according to claim 4, further comprising 5-fluorocytosine or an azole antifungal compound;
optionally, wherein the pharmaceutical composition is in a form of an injection preparation or an oral preparation.

15. The pharmaceutical composition according to claim 9, wherein:
the first active ingredient, the second active ingredient, and the third active ingredient are administered simultaneously or separately;
optionally, at least one of the first active ingredient, the second active ingredient, or the third active ingredient is in a form of an injection preparation or an oral preparation.

16. A method for prolonging half-life of amphotericin B or a salt thereof or improving an inhibitory activity of amphotericin B or a salt thereof or an inositol phosphorylceramide synthetase inhibitor against Cryptococcus neoformans, comprising administering the pharmaceutical composition according to claim 1.

17. The method according to claim 16, wherein said administering comprises simultaneously or separately administering the inositol phosphorylceramide synthase inhibitor and amphotericin B or the salt thereof;
optionally, wherein the inositol phosphorylceramide synthase inhibitor comprises a cyclic depsipeptide antibiotic;
optionally, wherein the cyclic depsipeptide antibiotic comprises at least one of aureobasidin A, enniatin, destruxin, emodepside, or fusafungine;
optionally, wherein the cyclic depsipeptide antibiotic comprises aureobasidin A;
optionally, wherein the salt of amphotericin B, if present, comprises at least one of: sulfate, phosphate, citrate, tartrate, fumarate, benzoate, adipate, succinate, mesylate, or maleate of amphotericin B;
optionally, wherein the cyclic depsipeptide antibiotic and amphotericin B or the salt thereof are administered in combination at a mass ratio ranging from 1:3 to 5:1;
optionally, wherein the cyclic depsipeptide antibiotic and amphotericin B or the salt thereof are administered in combination d at a mass ratio ranging from 1:2 to 4:1;
optionally, wherein the cyclic depsipeptide antibiotic and amphotericin B or the salt thereof are administered in combination at a mass ratio of 1:2, 1:1, 2:1, or 4:1;
optionally, wherein the co-administration further comprises administering at least one of 5-fluorocytosine or an azole antifungal compound;
optionally, wherein the at least one of 5-fluorocytosine or the azole antifungal compound, the cyclic depsipeptide antibiotic, and amphotericin B or the salt thereof are administered simultaneously or separately;
optionally, wherein at least one of the at least one of 5-fluorocytosine or the azole antifungal compound, the cyclic depsipeptide antibiotic, or amphotericin B or the salt thereof is in a form of an injection preparation or an oral preparation.

18. A method for treating or preventing a disease associated with Cryptococcus infection, comprising:
administering to a subject a pharmaceutically acceptable amount of the pharmaceutical composition according to claim 1;
optionally, wherein the disease associated with Cryptococcus infection comprises cryptococcal meningitis.
